(19) United States
(12) Patent Application Publication
(10) Pub. No.: US 2009/0222047 A1
(43) Pub. Date: Sep. 3, 2009

(54) Implant for correcting skeletal mechanics

(76) Inventor: Michael Graham, Shelby Township, MI (US)

Correspondence Address: KILE GOEKJIAN REED & MCMANUS, 1200 NEW HAMPSHIRE AVE, NW, SUITE 570, WASHINGTON, DC, 20036, US

(21) Appl. No.: 12/041,659

(22) Filed: Mar. 3, 2008

(51) Int. Cl. A61B 17/58

(52) U.S. Cl. 606/280

(57) ABSTRACT

Deformities present on the end of a bone, for example the end of the metatarsal bone making up part of the metatarsocuneiform joint, can lead to deformities such as bunions. These deformities are treatable with an implant that comprises a plate with a wedge extending perpendicular from the plate. Following removal of cartilage from the joint, deformed portions at the end of the bone are removed and the wedge is inserted in the joint and held in place when the plate is attached to the bones flanking the joint. This effectively fuses the two bones together. The wedge can be shaped in various ways depending on the particular deformity present.

### BACKGROUND OF THE INVENTION

This invention relates to a medical apparatus for enhancing and for correcting skeletal mechanics. More specifically, this invention relates to the correction of certain bone alignment deformities that impair optimal biped mechanics of the foot.

Excessive pronation (hyperpronation) of the foot leads to abnormal motion to the first metatarsal resulting in excessive strain on the soft tissues supporting this bone. After a prolonged period of these excessive forces the soft tissues will stretch out and no longer be able to support the first metatarsal. This instability leads to an abnormal deviation of the first metatarsal bone resulting in foot pathology. The deforming forces acting on the first metatarsal leads to three possible deviations: (1) pure medial deviation of the metatarsal, (2) dorsiflexion of the metatarsal, and (3) the combination of the previous two, dorsomedial deviation. When the first metatarsal deviates medially (toward the body's middle/away from the little toe) the hallux (big toe) will deviate laterally (toward the little toe). When the first metatarsal deviates dorsally (up), the proximal phalanx of the hallux plantarflexes (angles down) and the distal phalanx angles dorsally. A dorsomedially deviated first metatarsal forces the hallux to deviate in a plantar-lateral direction.

In addition to the soft tissue laxity of the first metatarsocuneiform joint there exists another deforming force. Several tendons from the leg and foot attach to the hallux. When the muscles from these tendons contract to move the hallux, this will lead to a retrograde force on the first metatarsal. The generated force from this action will further contribute to the deviation of the first metatarsal bone. The end result is that there will be instability at both the proximal and distal joints of the first metatarsal bone. To reiterate, the problem is not with the metatarsal bone itself but with the joints of this bone.

Yet, another complicating factor in the formation of first metatarsal bone (or “first ray”) deformities is the pathoanatomy of the end of the first or medial cuneiform bone. In a normal foot, the joint between the first metatarsal and first cuneiform should be straight across form medial to lateral. A common finding with first ray deformities is that the distal end of the cuneiform, the end in contact with the first metatarsal, is deformed. The end of the deformed cuneiform angles medially. In other words, the distal lateral end of the cuneiform is longer than the distal medial portion of the bone. This deformation causes instability of the first metatarsal bone and contributes to the medial shift of the metatarsal bone.

The deviation of the first metatarsal bone leads to the formation of a bunion or deformities of the first ray. These deformities include metatarsus primus elevatus, metatarsus primus varus, hallux abductovalgus, hallux limitus, hallux rigidus and metatarsus primus adductus. With all of these deformities, there is usually no actual intrinsic deformity of the first metatarsal itself. The deformities are proximal at the first metatarsal cuneiform joint or distal at the first metatarsophalangeal joint.

The current treatment of the deformity of the first ray ranges from conservative non-surgical to various surgical procedures. Non-surgical treatment includes the use of an arch support, supportive shoes, taping and strapping, padding, etc. Multiple surgical procedures have been described for the realignment of the first metatarsal bone to the cuneiform and the proximal phalanx of the hallux. These osseous, or bone surgical, procedures include cutting and shifting of the first metatarsal bone into a more rectus position to fusing the base of the first metatarsal bone to the first cuneiform.

The problem with non-surgical treatment options is that it is ineffective in eliminating the causative factor, meanwhile every step leads to further deformity of the metatarsal bone. The problem with this deformity is intrinsic so external remedies are ineffective in controlling the deforming forces.

Surgical remedies consist of various osseous procedures to realign the metatarsal bone to the hallux. These osseous procedures of the first metatarsal bone only provide for a cosmetic effect while the instability of the first metatarsal/first cuneiform joint still exists. These types of procedures straighten the metatarsal bone with respect to the hallux but leave instability at the first metatarsocuneiform joint. Since the instability at the first metatarsocuneiform joint still exists, the first metatarsal bone will eventually deviate again and lead, again, to the occurrence of the overall deformity.

Another surgical procedure has been suggested that consists of an opening wedge, or a bone graft, inserted between the first cuneiform and first metatarsal bones. Still another method to corrected this deformity of the first metatarsal bone was to fuse it to the first cuneiform via a surgical procedure. (The fusion of two bones, by surgical procedure or otherwise, is known as an arthrodesis.) These procedures lead to a long recovery periods, at least six months, and can fail. The wedge can displace from the fusion site. The bone graft can fail at a rate of 20-30%.

Another procedure is the shortening of the first metatarsal. Unfortunately, this transfers the body's weight to the second metatarsal head instead of the first metatarsal head. Often, further pathology ensues leading to callus formation under the ball of the foot leading to further pain, and possible ulceration. In normal ambulation, the weight of the body lands on the outer aspect of the heel and is transferred to through the foot ending up through the first metatarsal bone. The second metatarsal head is not meant to take the weight of the body and it is possible for it to develop a stress fracture.

Previously, various plates have been described for use in stabilizing the first metatarsal to the first cuneiform joint while the arthrodesis of these bones occurred. The problem with these plates is that they are use to stabilize the fusion site between these two bones and overall minic a screw or staple. There is still a shortening of the first metatarsal bone leading to the possibility of other ill effects. These plates are rather bulky and usually had to be removed after the arthrodesis of the two bones was achieved.

### BRIEF SUMMARY OF THE INVENTION

Surgery for deformity of the first ray (first metatarsal). This device consists of a plate with four screws and a triangular wedge.

### BRIEF DESCRIPTION OF THE FIGURES

FIG. 1 illustrates a preferred embodiment inserted into the patients left foot.

FIGS. 2A-C illustrate idealized geometric drawings for comparison of the wedge designs for each of the three deviations disclosed herein.

FIG. 3 illustrates a wedge for correcting pure medial deviations.

FIG. 4 illustrates a wedge for correcting dorsal deviations.

FIG. 5 illustrates a wedge for correcting dorsomedial deviations.

FIGS. 6-8 illustrate spacers used to identify the proper size wedge to be inserted.

### DETAILED DESCRIPTION OF PREFERRED EMBODIMENTS

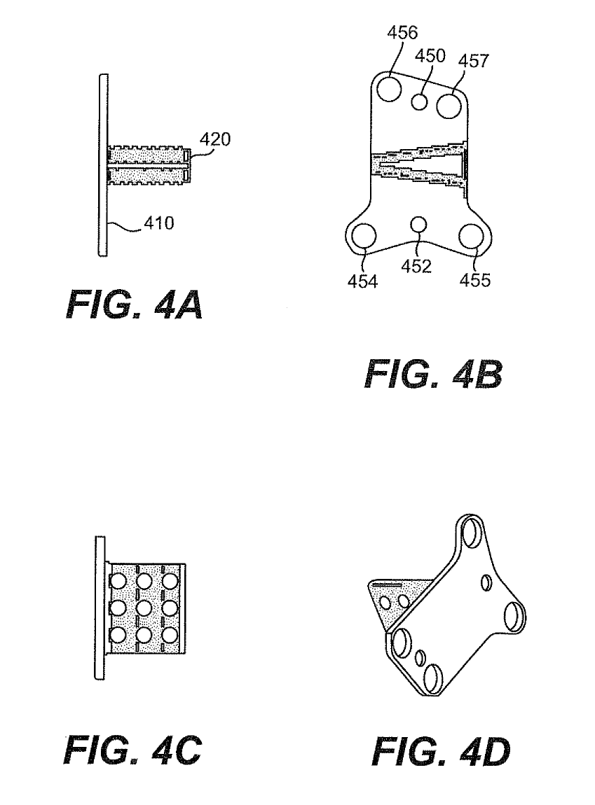

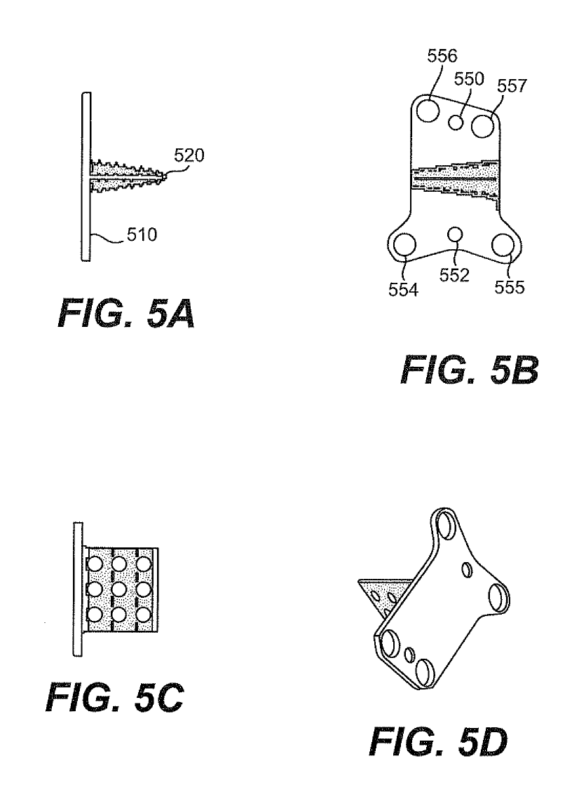

Turning to figure three, the plate has a proximal (back portion) and distal (front portion) end. The proximal wider end of the plate has two screw recesses 354, 355 for fixation of the plate in the first cuneiform. The wedge on the lateral side of the plate will be introduced between the first metatarsocuneiform joint to correct transfer of weight from the first cuneiform to the base of the first metatarsal bone. The distal narrower end of the plate also has two screw recesses 356, 357 for fixation onto the first metatarsal bone. Also found on the plate are two holes, one the proximal 352 and distal 350 segment for the first metatarsal and the first cuneiform bones. These holes are for temporary pin stabilization to hold the plate in place while the screws placed in the screw recesses (356, 357, 352, 350) to fixate the plate to the bones. See also similar screw recesses in FIGS. 4 and 5 (456, 457, 452, 450; 556, 557, 552, 550).

The interpositioned wedge will consist of various sizes to realign the first metatarsal back to where it is supposed to be. The length of the wedge has fenestrations that will allow bone to incorporate into the wedge to facilitate a fusion between the first metatarsocuneiform joint. The wedge will have a uniform taper with the widest part located at the base where it attaches to the plate extending to the tip the narrowest part.

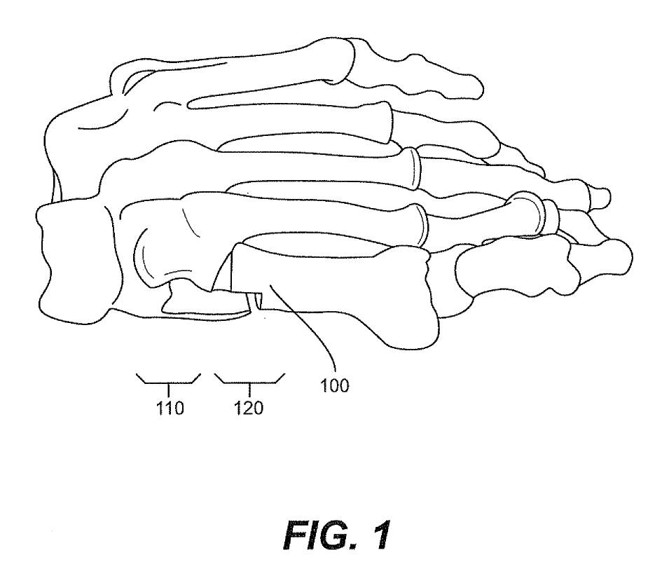

FIG. 1 is an illustration of the wedge properly placed on a users foot.

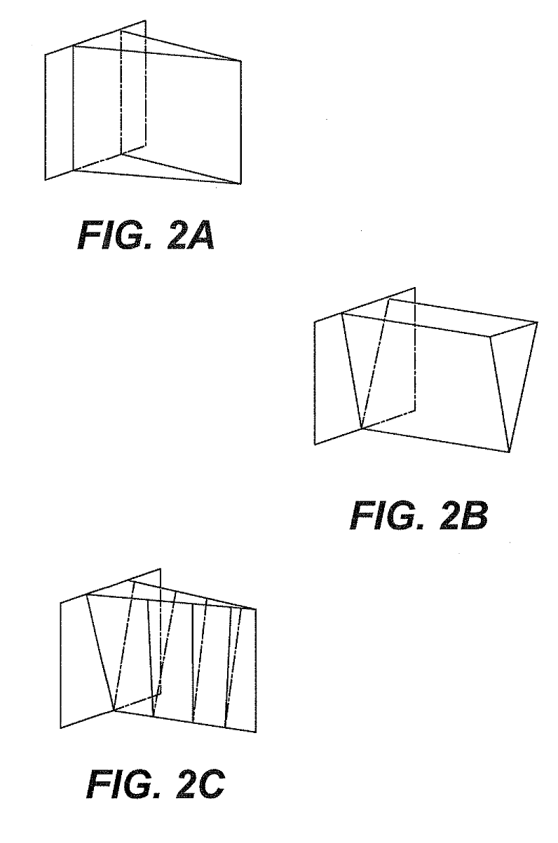

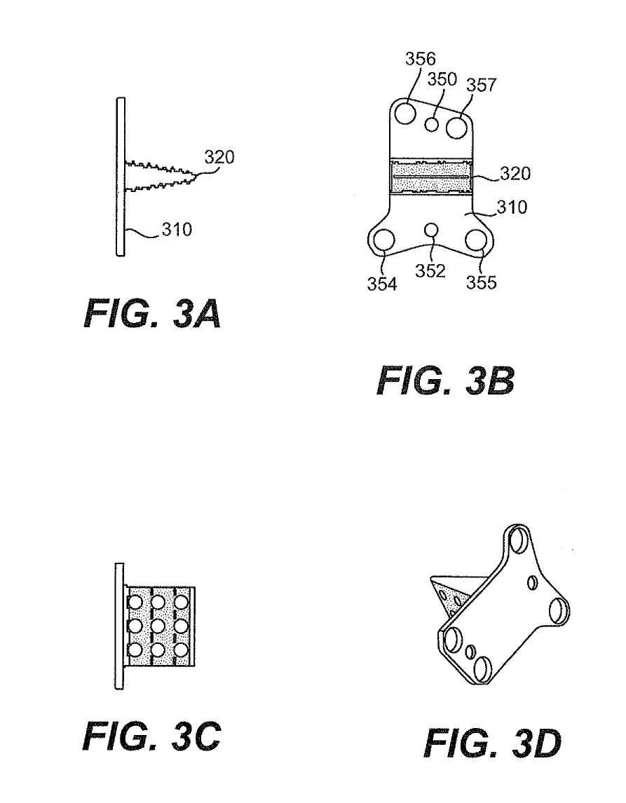

As we previously learned, there are three deviations of the first metatarsal bone, (1) medial, (2) dorsal, and (3) dorsomedial. Taking this into consideration, three different variations of the wedge are presented. These are illustrated in FIGS. 2-5. FIGS. 2A through 2C show idealized geometric figures for medial, dorsal, and dorsomedial variations, respectively. FIGS. 3-5 show preferred embodiments corresponding to each variation, respectively.

For the purely medially deviated joint, the wedge will be tapered uniformly from medial to lateral and dorsal to plantar on all sides. See FIG. 2A and FIG. 3.

For the dorsally deviated first metatarsal, the proximal part of the wedge will be flat against the first cuneiform, it will not be tapered. This distal part of the wedge will be angled with the wider part being on the dorsal aspect of the wedge and taper to the narrower part of the wedge being plantar. This is illustrated in FIG. 2A and FIG. 4.

The final overall design for the dorsomedially deviated first metatarsal consists of a wedge where the proximal end against the first cuneiform is not tapered from dorsal to plantar but the distal end against the first metatarsal is tapered. The narrowest part of the wedge is plantar and the wider part is dorsal and the overall attachment of the wedge to the plate is wider medially and tapers laterally.

Each of the three designed wedges will have incremental tapers to adjust for the severity of the deviation of the metatarsal. Trial wedges will be available, as part of the instrument set to determine the exact wedge needed for the correction of the deformity. Due to the design of the wedges they will be marked as right or left foot and what plane of deviation(s) it corrects.

The screws used to attach the plate to the first metatarsal and first cuneiform are self-tapping. The threaded head of the screw enters the plate so that it is flush. The part of the foot where the plate is to be placed does not have lots of soft tissues over it so a low profile is preferred.

The preferred material of the plate and wedge is titanium, however, other potential materials could also be used or combination of materials including ceramic, various bone graft compositions, polymers and the like.

### Insertion Procedure

A preferred insertion procedure is as follows. After the foot and ankle are prepped and draped in the usual fashion, a 4-cm linear incision is made over the first metatarsal first cuneiform joint. The soft tissue is dissected bluntly off these bones revealing the joint. The medial osseous prominence of the first cuneiform 110 is osteotomized to create a flush surface. A similar procedure is performed on the first metatarsal. 120. The lateral soft tissues of the joint are left intact.

The articular cartilage of the metatarsocuneiform joint is resected. One preferred method of cartilage removal is as follows. At the base of the first metatarsal a sagittal saw is used to remove the articular cartilage and also create a flush surface. At the distal aspect of the first cuneiform a sagittal saw is used to remove the articular cartilage present there. The goal is to remove as small amount of tissue as possible but enough so that there is osseous integration into the wedge.

Choosing the correct size wedge is important but will, most likely, not be possible before the operation. This is because the proper size will depend upon, not only the unique characteristics of the patients deformity, but also the surgeons decisions about how much tissue and bone to remove in the preceding preparatory steps.

Accordingly, the correct size wedge will be determined after the preceding preparatory steps with the aid of “trial sizers.” The various trial sizers are placed in between these two bones to determine which sized wedge will be needed to achieve the desired correction. The correction achieved with a specific trial wedge can be visualized under fluoroscopy or radiological intra-operative study.

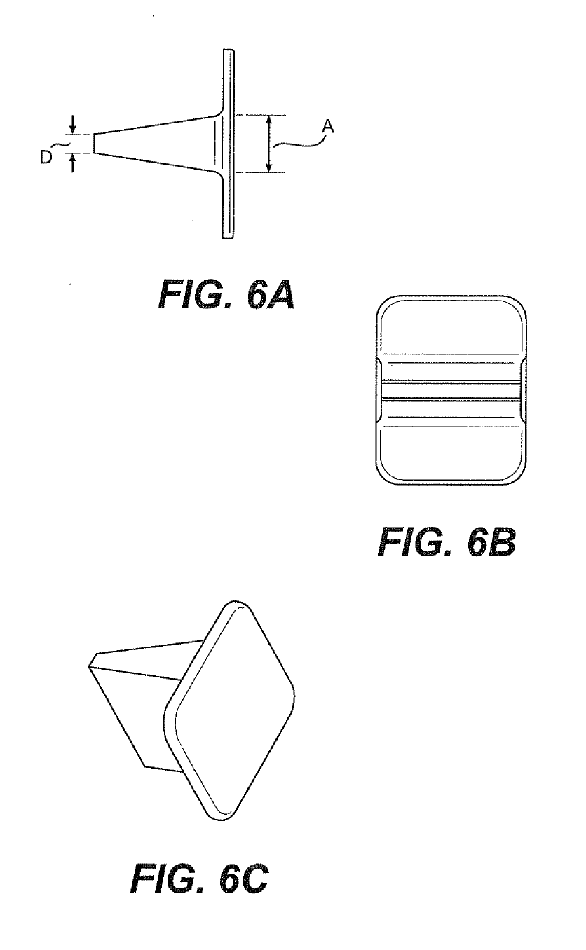

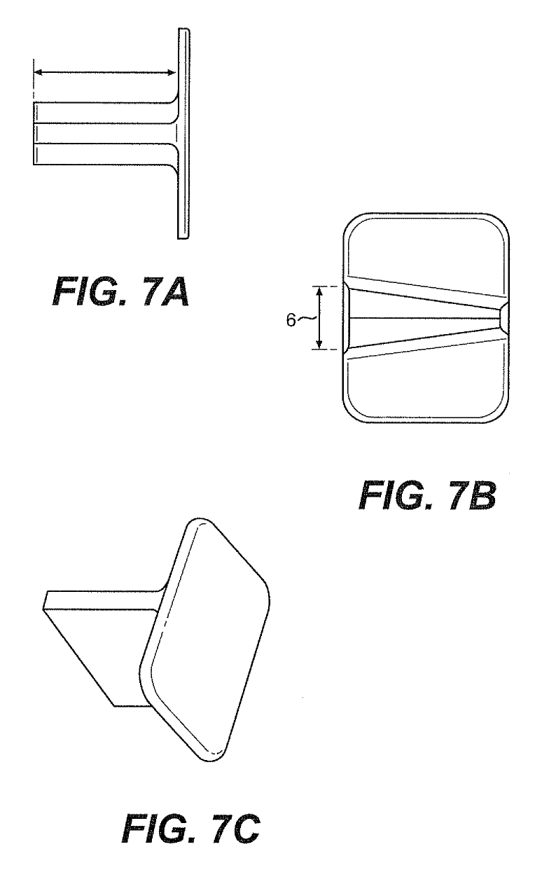

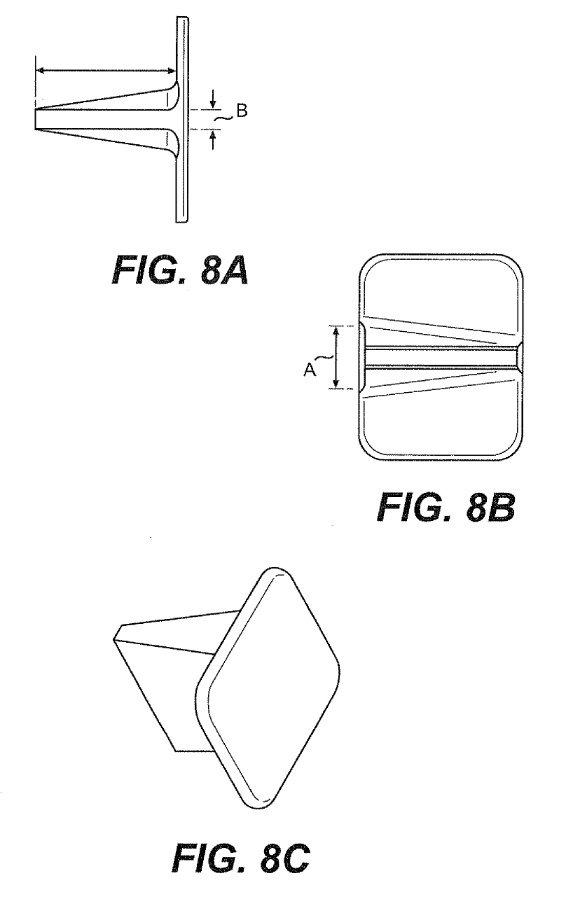

There can be a separate set of trial sizers designed for each of the three main classes of deviations mentions above: medial, dorsal and dorsomedial. These are illustrated in FIGS. 6-8 respectively.

Once the desired wedge size is determined it is placed into the joint space. A temporary pin is inserted first into the cuneiform to stabilize the plate while the screws will be inserted into the bones. A second stabilizing wire will then hold the end of the plate to the first metatarsal. Then the proximal screws are inserted into the cuneiform then the two screws are placed into the first metatarsal. The two temporary pins are then removed. The first metatarsal cuneiform joint is placed through a range of motion to visualize stabilization of the plate. Finally, radiographs or fluoroscopy are used to confirm the position of the screws and plate and the achieved correction.

Being completely satisfied with the results of the placement of the bone plate and wedge soft tissues and skin are closed per surgeon's choice. A dry sterile bandage is placed on the foot and the patient is allowed to ambulate with a surgical shoe.

The device presented here is an improvement over the predicate devices. For example, one prior art device is used to correct a defect or angular deviation of the first metatarsal bone. The current device, however, is not so limited in function. The current device could be used, not only on the first metatarsal bone, but also between the first metatarsal and cuneiform bones. Even though preferred embodiments herein have the indication for the first metatarsal cuneiform joint, other preferred embodiments of the device could be used in other areas of the foot as well as potentially the hand, or other locations in which similar pathologies exist as explained below.

Another advantage certain embodiments of the invention have over the prior art implants is that the prior art plates are attached in a somewhat tubular shape whereas embodiments of the current device can be flat. Also, prior art devices have an attached “bridge” which is flat and only extends a small amount whereas the inventor's device can have a much “longer” wedge.

Advantages of the inventor's device include stabilization of the first metatarsal bone with the first cuneiform that corrects the deviation of the first metatarsocuneiform joint while preventing the shortening of the first ray. Also, prior art arthrodesis procedures required a long recovery period, often about six weeks, before weight could be bone by the fused bones. In certain embodiments of the disclosed invention weight can be born after only 3 to 5 days. This is because unlike prior art procedures in which the area of bone fusion must heal before it can bear weight, the combination of the vertical plate and the wedge provide the structural support.

The device is not limited to the embodiments disclosed above. Other embodiments include any plate with an attached blade/wedge that has unique shape that can been used on the small bones of the hand, foot, wrist, ankle etc. Further, that there are several versions of the wedge, including one that corrects in the transverse plane, one that corrects in the sagittal plane, and one with the combination of both transverse and sagittal planes. Additionally, there are various degrees of correction achieved of each of the wedges depending on the thickness of the wedge.

### REFERENCES

Pinney, S J; Song, K R; Chou, L B; Surgical Treatment of Severe Hallux Valgus: The State of Practice Among Academic Foot and Ankle Surgeons. Foot Ankle Int. 27:1024-1029,
Munuera, P V; Dominguez, G; Polo, J; Rebollo, J; Medial Deviation of the First Metatarsal in Incipient Hallux Valgus Deformity. Foot Ankle Int. 27, 1030-1035, 2006
ElSain, A G; Tisdel, C; Donley, B; Sferra, J; Neth, D; Davis, B; First Metatarsal Bone: An Anatomic Study. Foot Ankle Int. 27, 1041-1048, 2006.
Madjarevic, M; et al. Mitchell and Wilson Metatarsal Osteotomies for the Treatment of Hallux Valgus Comparison of Outcomes Two Decades after the Surgery. Foot Ankle Int. 27, 887-893, 2006.
Grimes, J S; Coughlin, M J. First Metatarsophalangeal Arthrodesis as a Treatment for Failed Hallux Valgus Surgery. Foot Ankle Int., 27, 887-893, 2006.

What is claimed is:

1. A method of treatment of bone abnormalities by fusing two bones with an osteowedge comprising:
an osteowedge having a plate member and a wedge member,
said wedge member is connected to said plate member and extends perpendicularly from said plate member,
said wedge member is configured to insert in between said two bones,
said two bones and wedge member are secured together by securing said plate to both of said bones.
